(19) United States
(12) Patent Application Publication
(10) Pub. No.: US 2023/0381592 A1
(43) Pub. Date: Nov. 30, 2023

(54) NEUROLOGICAL REHABILITATION SYSTEM

(71) Applicant: Torque3, Inc., Centerville, UT (US)

(72) Inventors: David B. Ellzey, Centerville, UT (US); Boston Bradley Barham, Centerville, UT (US); Neven Šosteriè, Zagreb (HR); Antonio Toma{hacek over (s)}iæ, Karlovac (HR); Gordan Šikic, Medulin (HR); Charles Anderson Christopher, Salt Lake City, UT (US)

(21) Appl. No.: 17/825,818

(22) Filed: May 26, 2022

(51) Int. Cl. A63B 24/00; A63B 22/00; A63B 71/06; G16H 20/30; G16H 40/63; B25J 9/16

(52) U.S. Cl. CPC A63B 24/0087; A63B 22/0002; A63B 71/0622; A63B 24/0062; A63B 24/0075; G16H 20/30; G16H 40/63; B25J 9/1661; A63B 2022/0652

(57) ABSTRACT

The neurological rehabilitation system described includes at least visual display components, a robotic platform, a sensor array, and a neurological rehabilitation controller. The neurological rehabilitation controller controls the robotic platform based on outputs from the sensor array and generates at least a real-time visual simulation displayed using the visual display components. The generated real time visual simulation simulates a task as part of an enhanced task-oriented therapy for a patient undergoing neurological rehabilitation. The robotic platform is a physical structure that interfaces with the patient and facilitates the patient's movement of various body parts (e.g., arms and/or legs) in synchrony with the real-time visual simulation to perform a virtual task over time. The robotic platform also receives control instructions from the neurological rehabilitation controller, which articulate the platform and apply resistance force to the patient interface(s) to create a more realistic task experience.

### BACKGROUND

Neurological rehabilitation is a treatment regime for various nervous system injuries (e.g., stroke) and neurological diseases with the aim of treating physical and cognitive impairments caused by these ailments. One promising form of neurological rehabilitation treatment involves a task-oriented approach. Task-oriented training involves a patient performing various motor skills in a controlled environment, sometimes with the aid of a robotic device that includes components that move as instructed by computer software. It has been shown that beneficial neuroplastic changes in the cerebral cortex and in other parts of the central nervous system are linked to motor skill retraining in affected limbs. It is believed that motor skill retaining facilitates neural reorganization and “re-wiring” in the central nervous system. This ability of the central nervous system to re-wire itself is known as neuroplasticity.

### SUMMARY

This Summary is provided to introduce a selection of concepts, in a simplified form, that are further described hereafter in the Detailed Description. This Summary is not intended to identify key features or essential features of the claimed subject matter, nor is it intended to be used as an aid in determining the scope of the claimed subject matter.

Neurological rehabilitation system implementations described herein for treatment of nervous system injuries and neurological diseases generally include a visual component including a display, a robotic platform including a lower body apparatus that is contacted by a patient's left foot and right foot and that presents a resistive force which requires the patient to use lower body muscles to move the lower body apparatus with the patient's left foot and right foot, and a sensor array including at least one left leg sensor that measures the amount of force being applied by the patient's left leg to the lower body apparatus and at least one right leg sensor that measures the amount of force being applied by the patient's right leg to the lower body apparatus over time. In addition, the neurological rehabilitation system includes a neurological rehabilitation controller having one or more computing devices, and a neurological rehabilitation computer program having a plurality of sub-programs executable by the computing device or devices. The sub-programs configure the computing device or devices to control the amount of resistive force applied by the lower body apparatus to a left foot interface with the patient's left foot over time during a neurological rehabilitation session of the patient and control the amount of resistive force applied by the lower body apparatus to a right foot interface with the patient's right foot over time during the neurological rehabilitation session of the patient. In addition, sub-programs configure the computing device or devices to generate a real-time visual simulation of a task that is displayed to the patient via the visual component display over time during the neurological rehabilitation session of the patient based in part on the amount of force being applied by the patient's left foot to the left foot interface and right foot to the right foot interface.

Neurological rehabilitation system implementations described herein for treatment of nervous system injuries and neurological diseases also optionally have a robotic platform that includes an upper body apparatus that is contacted by a patient's left and right hands and that presents a resistive force which requires the patient to use upper body muscles to move the upper body apparatus with the patient's left and right hands, and have a sensor array that includes at least one left arm sensor that measures the amount of force being applied by the patient's left arm to the upper body apparatus and at least one right arm sensor that measures the amount of force being applied by the patient's right arm to the lower body apparatus over time. The optional implementations having an upper body apparatus, also have neurological rehabilitation computer program sub-programs that configure the computing device or devices to control the amount of resistive force applied by the upper body apparatus to a left hand interface with the patient's left hand over time during the neurological rehabilitation session of the patient and a right hand interface with the patient's right hand over time during the neurological rehabilitation session of the patient. In addition, sub-programs configure the computing device or devices to generate a real-time visual simulation of a task that is displayed to the patient via the visual component display over time during the neurological rehabilitation session of the patient based in part on the amount of force being applied by the patient's left and right hands to the left and right hand interfaces.

Some neurological rehabilitation system implementations described herein for treatment of nervous system injuries and neurological diseases have an upper body apparatus, but no lower body apparatus. These implementations generally include a visual component including a display, a robotic platform including an upper body apparatus that is contacted by a patient's left and right hands and that presents a resistive force which requires the patient to use upper body muscles to move the upper body apparatus with the patient's left and right hands, and a sensor array including at least one left arm sensor that measures the amount of force being applied by the patient's left arm to the upper body apparatus and at least one right arm sensor that measures the amount of force being applied by the patient's right arm to the lower body apparatus over time. In addition, the neurological rehabilitation system includes a neurological rehabilitation controller having one or more computing devices, and a neurological rehabilitation computer program having a plurality of sub-programs executable by the computing device or devices. The sub-programs configure the computing device or devices to control the amount of resistive force applied by the upper body apparatus to a left hand interface with the patient's left hand over time during a neurological rehabilitation session of the patient and a right hand interface with the patient's right hand over time during the neurological rehabilitation session of the patient. In addition, sub-programs configure the computing device or devices to generate a real-time visual simulation of a task that is displayed to the patient via the visual component display over time during the neurological rehabilitation session of the patient based in part on the amount of force being applied by the patient's left and right hands to the left and right hand interfaces.

### DESCRIPTION OF THE DRAWINGS

The specific features, aspects, and advantages of the neurological rehabilitation system implementations described herein will become better understood with regard to the following description, appended claims, and accompanying drawings where:

FIG. 1 is a diagram illustrating an exemplary implementation, in simplified form, of the neurological rehabilitation system implementations described herein.

FIGS. 2A-B are diagrams illustrating an exemplary implementation, in simplified form, of a quadricycle neurological rehabilitation platform shown from the right side (FIG. 2A) and the left side (FIG. 2B).

FIG. 3 is a diagram illustrating an exemplary implementation, in simplified form, of an overboot accessory for the quadricycle neurological rehabilitation platform.

FIG. 4 is a diagram illustrating an exemplary implementation, in simplified form, of a synchronized support rail (SSR) accessory for the quadricycle neurological rehabilitation platform.

FIG. 5 is a diagram illustrating an exemplary implementation, in simplified form, of a transfer seatbelt accessory for the quadricycle neurological rehabilitation platform.

FIG. 6 is a diagram illustrating one implementation, in simplified form, of various adaptation sub-programs.

FIG. 7 is a diagram illustrating one implementation, in simplified form, of an enhanced task-oriented therapy concept.

FIG. 8 is a diagram illustrating a simplified example of a general-purpose computer system on which various implementations and elements of the neurological rehabilitation system, as described herein, may be realized.

### DETAILED DESCRIPTION

In the following description reference is made to the accompanying drawings which form a part hereof, and in which are shown, by way of illustration, specific implementations in which a neurological rehabilitation system can be practiced. It is understood that other implementations can be utilized, and structural changes can be made without departing from the scope of the neurological rehabilitation system.

It is also noted that for the sake of clarity specific terminology will be resorted to in describing the neurological rehabilitation system implementations described herein and it is not intended for these implementations to be limited to the specific terms so chosen. Furthermore, it is to be understood that each specific term includes all its technical equivalents that operate in a broadly similar manner to achieve a similar purpose. Reference herein to “one implementation”, or “another implementation”, or an “exemplary implementation”, or an “alternate implementation”, or “some implementations”, or “one tested implementation”; or “one version”, or “another version”, or an “exemplary version”, or an “alternate version”, or “some versions”, or “one tested version”; or “one variant”, or “another variant”, or an “exemplary variant”, or an “alternate variant”, or “some variants”, or “one tested variant”; means that a particular feature, a particular structure, or particular characteristics described in connection with the implementation/version/variant can be included in one or more implementations of the neurological rehabilitation system. The appearances of the phrases “in one implementation”, “in another implementation”, “in an exemplary implementation”, “in an alternate implementation”, “in some implementations”, “in one tested implementation”; “in one version”, “in another version”, “in an exemplary version”, “in an alternate version”, “in some versions”, “in one tested version”; “in one variant”, “in another variant”, “in an exemplary variant”, “in an alternate variant”, “in some variants” and “in one tested variant”; in various places in the specification are not necessarily all referring to the same implementation/version/variant, nor are separate or alternative implementations/versions/variants mutually exclusive of other implementations/versions/variants. Yet furthermore, the order of process flow representing one or more implementations, or versions, or variants does not inherently indicate any particular order nor imply any limitations of the neurological rehabilitation system.

As utilized herein, the terms “module”, “component,” “system,” “client” and the like can refer to a computer-related entity, either hardware, software (e.g., in execution), firmware, or a combination thereof. For example, a component can be a process running on a processor, an object, an executable, a program, a function, a library, a subroutine, a computer, or a combination of software and hardware. By way of illustration, both an application running on a server and the server can be a component. One or more components can reside within a process and a component can be localized on one computer and/or distributed between two or more computers. The term “processor” is generally understood to refer to a hardware component, such as a processing unit of a computer system.

Furthermore, to the extent that the terms “includes,” “including,” “has,” “contains,” and variants thereof, and other similar words are used in either this detailed description or the claims, these terms are intended to be inclusive, in a manner similar to the term “comprising”, as an open transition word without precluding any additional or other elements.

### 1.0 Introduction

In general, the neurological rehabilitation system implementations described herein for treatment of nervous system injuries and neurological diseases include visual display components, a robotic platform, a sensor array, and a neurological rehabilitation controller. In addition, in some implementations, other simulated feedback elements are included to provide a more realistic experience to the patient. For example, in addition to the visual display components, spatial audio components and scent producing components can be included to mimic the sounds and smells a patient would experience during real tasks. The neurological rehabilitation controller controls the robotic platform based on outputs from the sensor array and generates a real-time visual simulation displayed to the user using the visual display components. The generated real-time visual simulation simulates a task as part of a task-oriented therapy for a patient undergoing neurological rehabilitation, which allows the patient to navigate through a virtual world with human-powered locomotion using the robotic platform. The robotic platform is a physical structure that interfaces with the patient and facilitates the patient's movement of various body parts (e.g., arms and/or legs) in synch with the real-time visual simulation to perform a virtual task over time. The robotic platform also receives control instructions from the neurological rehabilitation controller, which articulate the platform and apply resistance force to the patient interface(s) to create a more realistic task experience. For example, the robotic platform can be articulated in ways that simulate the feeling of turning the human-powered vehicle or riding up or down a hill. The robotic platform can also be articulated to simulate the gravity forces (g-forces) and inertia a patient would experience moving through the real world on a human-powered vehicle. Further, the robotic platform can be articulated to simulate the vibrations of tires on a path or roadway, the bumps and impacts felt by a rider of a human-powered vehicle while traversing the real world, and even the force of the wind pushing against the human-powered vehicle. The resistance force applied to the patient interface(s) constantly changes over the course of a neurological rehabilitation session to simulate the resistance that would be felt by a rider of a human-powered vehicle as they move through the world. For example, the resistance would increase when going up hills and decrease when going down a hill. The resistance could also vary to simulate the type of surface the human-powered vehicle is traversing. For example, greater resistance would be experience when moving along a gravel path, than it would moving on a paved roadway.

The neurological rehabilitation system implementations described herein have many advantageous uses. For example, the immersive experience provided during a neurological rehabilitation session and the task-oriented therapy improve a patient's neuroplasticity. This is advantageous for patients with a brain injury (such as stroke), as well as degenerative brain and nervous system diseases. It is also believed that this type of task-oriented training can be beneficial to a patient for neurological behavioral training (including for autistic persons). Further, this type of therapy can be beneficial to seniors for general neurological upkeep. There is also a possibility that this type of therapy can be useful in pain management.

Further, it has been found that an enhanced task-oriented therapy utilizing virtual reality and robotic assistance to simulate tasks that are continually challenging and continually novel with meaningful rewards result in more successful treatments. The neurological rehabilitation system implementations described herein achieve this goal by delivering the illusion of presence and sense of embodiment required for a patient to achieve deep immersion in a task. This deep immersion allows a patient to suspend their disbelief that they are simply in a simulation, which allows a perception of risk to be injected into the simulated task. This, in turn, creates two important capabilities. First, it adds intensity to the overall experience which unto itself can improve neuroplasticity but more importantly, it allows using the perception of risk to assign urgency and importance to the actions performed during task-oriented therapy resulting in a novel enhanced task-oriented therapy. This enhanced task-oriented therapy will be described in more detail in sections to follow.

### 2.0 Neurological Rehabilitation System

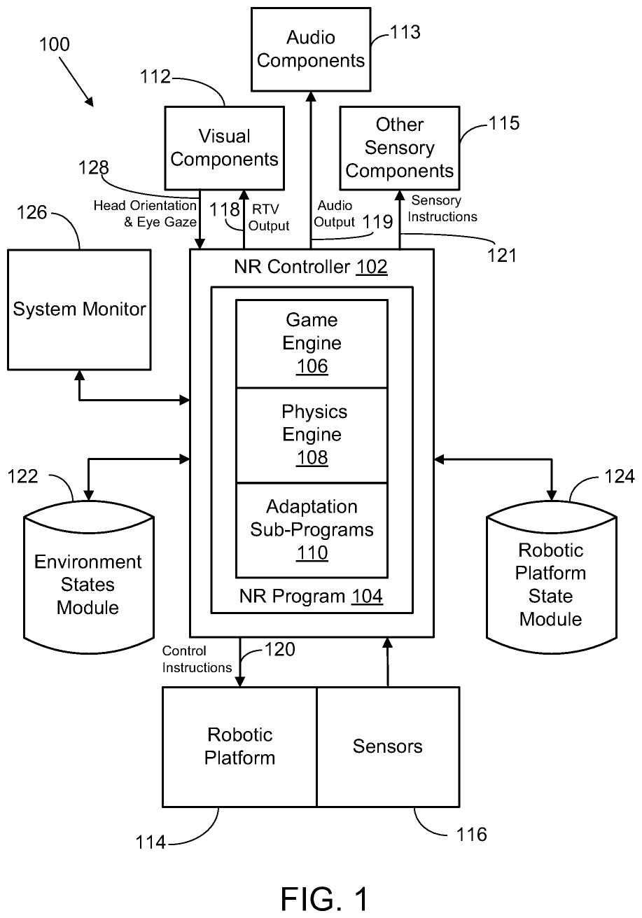

FIG. 1 illustrates an exemplary system diagram for the neurological rehabilitation system implementations described herein. In particular, the system diagram of FIG. 1 illustrates the interrelationships between various elements of the neurological rehabilitation system. While the system diagram of FIG. 1 illustrates a high-level view of the neurological rehabilitation system implementations described herein it is not intended to provide an exhaustive or complete illustration of every possible implementation, nor is it intended to suggest any limitation as to the scope of use or functionality of the neurological rehabilitation system.

As exemplified in FIG. 1, the neurological rehabilitation system 100 includes the following components. At the heart of the system is a neurological rehabilitation (NR) controller 102 made up of one or more computing devices (such as those described in Section 3.0 of this description), and a neurological rehabilitation (NR) computer program 104 that includes a plurality of sub-programs executable by the computing device or devices of the controller. For convenience in describing the neurological rehabilitation computer program 104, it can be thought of as being made up of three sub-program categories—namely a game engine 106, its integrated physics engine 108, and a suite of adaptation sub-programs 110 that adapt an existing game engine and physics engine package to function as the neurological rehabilitation controller. In the context of the neurological rehabilitation system implementations described herein, the game engine and its integrated physics engine are used to create a real-time visual simulation of a task that is displayed to a patient during a rehabilitation session. The specifics of the task and how it unfolds over time are controlled by the suite of adaptation sub-programs 110.

The neurological rehabilitation system implementations described herein also include visual display components 112, a robotic platform 114 and a sensor array 116. In general, the visual display components 112 are used to display a real-time visual simulation of a task to a patient undergoing neurological rehabilitation during a rehabilitation session. To this end, the real-time visual output 118 from the neurological rehabilitation controller 102 is output to the visual display components 112. The robotic platform 114 is a physical structure that interfaces with the patient and facilitates the patient's movement of various body parts (e.g., arms and/or legs) in synch with the real-time visual simulation to perform a virtual task over time. The robotic platform 114 also receives control instructions 120 from the neurological rehabilitation controller 102, which are implemented to articulate the platform and apply resistance force to the patient interface(s) to create a more realistic task experience. The sensors 116, which can be integrated into the robotic platform 114, are used to monitor the results of the patient's movements as well as the orientation of the robotic platform.

The neurological rehabilitation controller 102 also has access to, and is fed information from, various libraries. More particularly, an environmental state library 122 provides various environmental states that inform the neurological rehabilitation controller 102 of simulated environmental conditions applicable to the simulated task over time (e.g., the environmental conditions change as the simulated task progresses). These environmental states are employed in the generation of the simulated real-time visualization 118 of the task as well as in the control of the robotic platform 114. A platform operation instruction library 124 is also accessible, and feeds information to the neurological rehabilitation controller 102. For example, the platform operation instruction library 124 can include instructions that inform the neurological rehabilitation controller 102 on how to orient the robotic platform 114 or how much resistance is to be applied to the patient interface(s) when certain conditions in the simulated task are encountered.

Some implementations of the neurological rehabilitation system 100 also include a system monitor 126 (as shown in the implementation depicted in FIG. 1). In one implementation, the system monitor 126 is a computer workstation that includes at least one display and various user input devices. The system monitor 126 is in two-way communication with the neurological rehabilitation controller 102. The controller 102 transfers information about a current rehabilitation session to the system monitor 126, which the monitor displays to a user such as a rehabilitation therapist. In one implementation, the user can also input data and commands into the system monitor 126 for transfer to the neurological rehabilitation controller 102.

More detailed descriptions of the neurological rehabilitation system components are provided in the sections to follow. To this end, while the neurological rehabilitation system can take many forms, the descriptions to follow will feature implementations in the form of a recumbent stationary quadricycle with blades for upper body training, but with the upper body training blades adapted to provide a virtual steering capability. For the purposes of this description the foregoing recumbent stationary quadricycle will be referred to simply as a “quadricycle”, and the upper body training blades with virtual steering capability will be referred to as “steering blades”. In the case of a quadricycle example, the patient may use their arms and legs to move pedals and steering blades to virtually pedal and steer the quadricycle along a path depicted in a real-time visual simulation. The neurological rehabilitation system could take a similar but simpler form where the quadricycle only requires the patient to use their arms, or only use their legs, to simulate locomotion in a real-time visual simulation of a bike riding task.

### 2.1 Visual, Audio and Other Sensory Components

The visual display components 112 of the neurological rehabilitation system 100 can take several different forms. In one version, the visual display components 112 take the form of a Virtual Reality (VR) headset. The VR headset is mounted on the patient's head and has stereoscopic displays providing separate images to each eye which together create a virtual environment. In the context of a quadricycle, the virtual environment mimics what a rider would see while riding along a course in a designed activity—such as a bike trail. The VR headset also includes internal sensors that track head motion and eye gaze direction. As shown in FIG. 1, this information 128 is fed back to the neurological rehabilitation controller 102 which uses it to modify the real-time visualization output 118 on nearly a real time basis to change the field of vision of the virtual environment to match the patient's current head orientation and eye gaze direction.

In another version of the visual display components, the VR headset is replaced with one or more conventional display screens which display the virtual environment.

A VR headset or conventional display setup can also include audio components 113 for audio playback. For example, the audio components can include one or more loudspeakers (e.g., a VR headset typically includes loudspeakers speakers or an integrated headphone/earphone apparatus to provide a spatial audio playback to the wearer). The neurological rehabilitation controller 102 would generate and output an audio output 119 to go along with the real-time visualization output 118. The audio output 119 would be played to the patient during the rehabilitation session. For example, the audio could mimic the ambient sounds that would typically be heard by a quadricycle rider riding along a real trail, such as sounds like the quadricycle chain would make, or the sound of the tires as they roll over a wooden bridge or other road surface. In general, any sound that mimics interactions between a quadricycle ridden outdoors and the environment, could be simulated, synchronized with the real-time visualization and played during the virtual ride.

Other sensory components 115 can also be included. For example, a scent producing component could be incorporated to mimic the environmental smells a patient would experience during an outdoor quadricycle ride. The scent of trees synchronized with a simulated ride through a forest, or the scent of the ocean synchronized with a simulated ride along the seashore, and so on, may enhance a neurological rehabilitation session experience. The neurological rehabilitation controller 102 would generate and output sensory instructions 121 to go along with the real-time visualization output 118. The sensory instructions 121 would be executed by the sensory components 115 during the rehabilitation session.

### 2.2 Neurological Rehabilitation Robotic Platform

In general, the neurological rehabilitation robotic platform has a lower body apparatus that is contacted by a patient's left and right feet and that presents a resistive force which requires the patient to use lower body muscles to move with their feet. In addition, some implementations of the robotic platform have an upper body apparatus that is contacted by a patient's left and right hands and that presents a resistive force which requires the patient to use upper body muscles to move with their hands. Still further, some implementations of the robotic platform have both the lower and upper body apparatuses.

More particularly, in some implementations, the neurological rehabilitation robotic platform is a stationary human-powered recumbent virtual quadricycle having a left foot interface that takes the form of a left-side pedal, and the right foot interface that takes the form of right-side pedal. The left and right-side pedals are attached to the distal end of left and right-side crank arms, respectively, and the proximal ends of the crank arms are attached to a common crank axle (as will be described in more detail below). The cranks project in a perpendicular direction from the crank axle and in opposite directions from each other. The resistive force applied to the left and right-side pedals is applied by a pedal servo motor of the lower body apparatus.

In some implementations, the neurological rehabilitation robotic platform is a stationary human-powered recumbent virtual quadricycle having a left hand interface that takes the form of left-side steering blade, and a right hand interface that takes the form of right-side steering blade. In these implementations, the resistive force applied to the left-side and right-side steering blades is applied by a steering blade servo motor of the upper body apparatus.

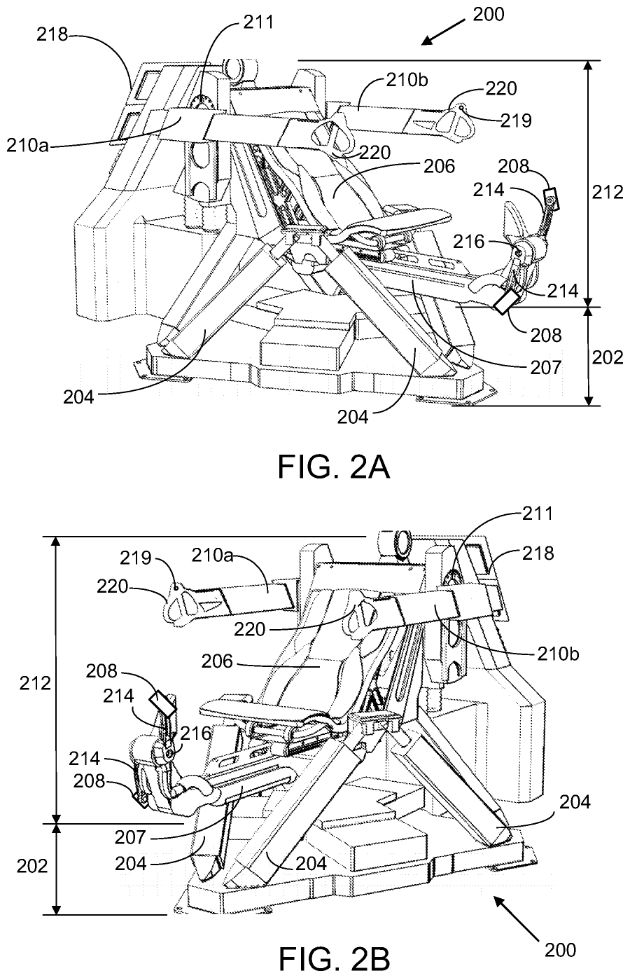

In some implementations, the neurological rehabilitation robotic platform is the previously described quadricycle that includes both the foregoing left and right foot interfaces, as well as the left and right hand interfaces. FIGS. 2A-B depict an exemplary version of the quadricycle neurological rehabilitation platform 200 shown from the right side (FIG. 2A) and the left side (FIG. 2B). It is noted that the configuration of the platform 200 shown in FIGS. 2A-B is not intended to represent the only configuration possible. Generally, in this exemplary implementation, any configuration that includes an articulating base 202 (with articulation mechanisms 204), a patient seat 206, a pair of pedals 208, a pair of steering blades 210a, 210b, servo motors (not shown) one of which is connected to the pedals and one to the blades, and various sensors (not shown) that sense the current conditions of foregoing components, would be acceptable.

### 2.2.1 Articulating Base

The robotic platform 200 can be characterized as having a patient portion 212 that accommodates the patient and an articulating base 202 that attaches to the patient portion via one or more articulation mechanisms 204. The articulating base 202 and its articulation mechanism(s) 204 moves the patient portion 212 of the robotic platform to produce various pitch or roll conditions, or combinations thereof, to enhance the perceived realism of the riding experience. In one implementation, the base 202 moves the patient portion 212 to impart simulated gravitational forces (g-forces) to the patient. For example, if the patient makes a virtual hard right turn, the base 202 leans them left so that gravity makes them feel as if they are being pulled in the opposite direction of their turn. The same process applies for accelerating and decelerating (e.g., braking) by leaning the patient portion and so the patient back and forward respectively. The base 202 will also vibrate the patient portion 212 to mimic a real quadricycle as it moves across various surfaces. For example, the vibration will be low level and constant when the course being simulated is a smooth surface such as a paved road, whereas the vibration will be rougher and random to simulate a rough trail such as a dirt or gravel road, or an offroad condition. Still further, the base 202 will impart quick up-down movements to the patient portion 212 to mimic the bounce of a quadricycle ridden outdoors when it hits a rise or dip in the trail.

### 2.2.2 Patient Seat and Pedal Boom

The patient seat 206 of the neurological rehabilitation robotic platform depicted in FIGS. 2A-B is a recumbent seat that allows the patient to sit in a comfortable reclined position with their legs extending forward toward the aforementioned pedals 208. The seat 206 can be adjusted forward for ease of access when the patient enters the patient portion 212 of the robotic platform 200, and then returned to its operating position prior to operation. It is important to return the seat 206 to its operating position because the center of gravity of the patient portion 212 should be as close to center of the robotic platform 200 as possible. The patient seat 206 can also include safety belts or harnesses (not shown) to hold the patient safely in the seat during a rehabilitation session.

A pedal boom 207 depicted in FIGS. 2A-B is extendable and retractable so that the pedals 208 can be set in a position where the patient's feet are on the pedals and their legs can extend to the furthest reach of the pedals as they are rotated through their cycle.

### 2.2.3 Pedals and Pedal Servo Motor

In one implementation, the pedals 208 employed on the neurological rehabilitation robotic platform are conventional bicycle pedals. These pedals can also advantageously include a retaining strap or structure (not shown) that holds the patient's foot and allows the patient to move the pedal with a pulling motion rather than just a pushing motion. Each pedal 208 is rotatably attached to the distal end of a crank arm 214 and generally extend in a perpendicular direction from the arm. The crank arms 214 are attached at their proximal ends to opposite sides of a pedal axle 216 and generally extend perpendicular to the common pedal axle and in opposite directions. The pedals 208 operate like the pedals on a conventional quadricycle, except that instead of rotating a crank to power the quadricycle, the pedals operate to rotate a pedal servo motor (not shown) that is attached to the pedal axle 216 on the neurological rehabilitation robotic platform 200. The servo motor is controlled by the neurological rehabilitation controller (102 in FIG. 1 and exemplified by 218 in FIGS. 2A-B) and used to create a resistance force on the pedal axel that the patient must overcome to move the pedals 208.

### 2.2.4 Steering Blades and Steering Blade Servo Motors

In one implementation, the steering blades 210a, 210b employed on the neurological rehabilitation robotic platform 200 are cantilevered beams having an adjustable length and hand grips 220 located at their distal ends. The proximal end of each steering blade 210a, 210b is attached to a steering blade axle mechanism 211 that extends laterally across the patient portion of the robotic platform between the left and right steering blades. The steering blade axle mechanism 211 has a counter-rotating right-angle gearbox (not shown) located between the steering blades. In one version, this gearbox has a T-shape with two shafts that extend outward along the same axis (in this case extending to the proximal ends of the left and right steering blades, respectively), and a third shaft extending at a right angle from the axis of the first two shafts and which when rotated turns the other two shafts, and vice versa. The gearbox is counter-rotating so that when one of the first two shafts rotates in a counterclockwise direction, the other of the first two shafts rotates clockwise, and vice versa. A steering blade servo motor (not shown) is attached to the third shaft and is used to create a resistance that must be overcome by rotating the first or second shafts, or as will be discussed shortly, rotating both the first and second shafts at the same time in opposite rotational directions. The result of using the steering blade axle mechanism 211 is that the steering blades 210a, 210b are interlocked laterally to provide a more natural steering experience where a patient pulls down with one arm while pushing up with the other to turn the virtual quadricycle. This up-down motion is designed to engage a patient's upper body skeletal-muscular system to steer the virtual quadricycle during a neurological rehabilitation session and is thought to enhance and increase neuroplasticity.

The steering blades 210a, 210b extend forward from their attachment to the steering blade axle mechanism 211 and in a neutral position (i.e., the position where the blades are not turning the virtual quadricycle to the left or right) are horizontally level. In one implementation, an upward motion of steering blade 210b located on the left side of the patient and corresponding downward motion of the steering blade 210a located on the right side of the patient, simulates a right-hand turn. Conversely, an upward motion of steering blade 210a located on the right side of the patient and corresponding downward motion of the steering blade 210b located on the left side of the patient, simulates a left-hand turn. The extent of the up-down motion of the blades 210a, 210b dictates the degree of the simulated turn. Alternate implementations can employ a different turning scheme where an upward motion of steering blade 210b located on the left side of the patient and corresponding downward motion of the steering blade 210a located on the right side of the patient, simulates a left-hand turn, and an upward motion of steering blade 210a located on the right side of the patient and corresponding downward motion of the steering blade 210b located on the left side of the patient, simulates a right-hand turn. Each steering blade 210a, 210b is adjustable in length, and can be adjusted in height at their proximal ends to accommodate the patient. When adjusted properly, the patient should be able to extend their arms to grip the hand grips 220 with the steering blades 210a, 210b being about shoulder height.

The steering blade servo motor attached to steering blade axle mechanism 211 is controlled by the neurological rehabilitation controller (102 in FIG. 1 and exemplified by 218 in FIGS. 2A-B) and used to create a resistance force on the blades that the patient must overcome to move the blades.

It is noted that other steering mechanisms for the neurological rehabilitation system described herein are also possible and it is not intended to limit the neurological rehabilitation robotic platform 200 to just the implementations of the steering blades described above. For example, a steering lever arrangement could be used in which the levers extend forward similar to the above-described steering blades, but at hip level instead of shoulder level. In this alternate implementation, the hip level steering levers operate in the same manner as the steering blade implementations describe above, except that the patient would reach down and grasp grips at the distal ends of the levers located on either side of their legs. Another example of an alternate steering mechanism is a traditional steering wheel arrangement where the patient would grasp a steering wheel and rotate it clockwise or counterclockwise to perform a virtual turn of the simulated quadricycle to the right or left, respectively.

### 2.2.5 Brake Feature

In one implementation, the neurological rehabilitation system includes a brake feature that allows a patient to apply the brakes of the virtual quadricycle and see it slow down in the displayed real-time visual simulation as well as feel the deceleration via articulations of the robotic platform as described previously. One version of the brake feature involves the inclusion of a brake button (219 in FIGS. 2A-B) on one or both of the hand grips 220 of the steering blades 210a, 210b. When the patient activates one or both of the brake buttons 219, a brake activation signal is sent to the neurological rehabilitation controller (102 in FIG. 1) along with the current sensor readings at each time step for as long as the brake button(s) remain activated. The adaptation sub-programs 110 of the neurological rehabilitation computer program 104 receive the brake activation signal and causes the simulated quadricycle to reduce speed in the real-time visual simulation a prescribed amount for each time step the brake button remains activated. In addition, in one implementation, the adaptation sub-programs 110 cause the articulation of the robotic platform in a way that mimics what a patient riding a real quadricycle would feel its brakes are applied. For example, the patient portion 212 of the robotic platform could be tilted forward to simulate the quadricycle braking. It is noted that other brake feature activation methods are also possible. For example, an activation method involving rotating the pedals in a reverse direction could be used to activate the brake feature mimicking how a coaster brake works on a real bicycle.

### 2.2.6 Sensor Array

As indicated previously and shown in FIG. 1, the neurological rehabilitation system 100 includes various sensors 116 that sense the current conditions of the various components of the robotic platform 114, and in one implementation the patient as well (e.g., heart rate, breathing rate, skin conductance, and so on). While external sensors (e.g., cameras) could be used as one of more of the sensors, in one implementation, the sensors are incorporated into the robotic platform itself.

More particularly, in one implementation, the sensor array includes at least one left leg sensor that measures the amount of force being applied by the patient's left leg to the left pedal and at least one right leg sensor that measures the amount of force being applied by the patient's right leg to the right pedal. In one implementation, the left leg and right leg sensors are incorporated into the left and right foot interfaces, respectively. In one version, the sensor array also includes a sensor in the pedal servo motor to sense the rotation speed of the pedals and the rotational position of the servo motor axle. Still further, in one version, the sensor array includes a sensor in the pedal servo motor that senses the resistance to rotation being applied by the pedal servo motor.

In one implementation, the sensor array further includes at least one left arm sensor that measures the amount of force being applied by the patient's left arm to the left steering blade in either the upward or downward direction, and at least one right arm sensor that measures the amount of force being applied by the patient's right arm to the right steering blade in either the upward or downward direction. In one implementation, the left arm and right arm sensors are incorporated into the left and right arm interfaces, respectively. In one version, the sensor array also includes sensors in the steering blade servo motor to sense the rotation speed and rotational position of the steering blade servo motor. Still further, in one version, the sensor array included sensors in the steering blade servo motor that sense the resistance to rotation being applied by the steering blade servo motor.

In one implementation, the sensor array further includes sensors that sense the position of each of the robotic platform base's articulation mechanisms. The signals from these sensors alone can be used to compute the orientation of the patient portion of the robotic platform, or sensors that sense the orientation of the patient portion can be included to make the calculation, or both.

Each sensor in the sensor array is in communication with the neurological rehabilitation controller and the signal output from each sensor is sent to the controller. Once received, the sensor signals are processed and transmitted to the neurological rehabilitation computer program for use as described previously and as will be described next.

### 2.2.7 Overboot

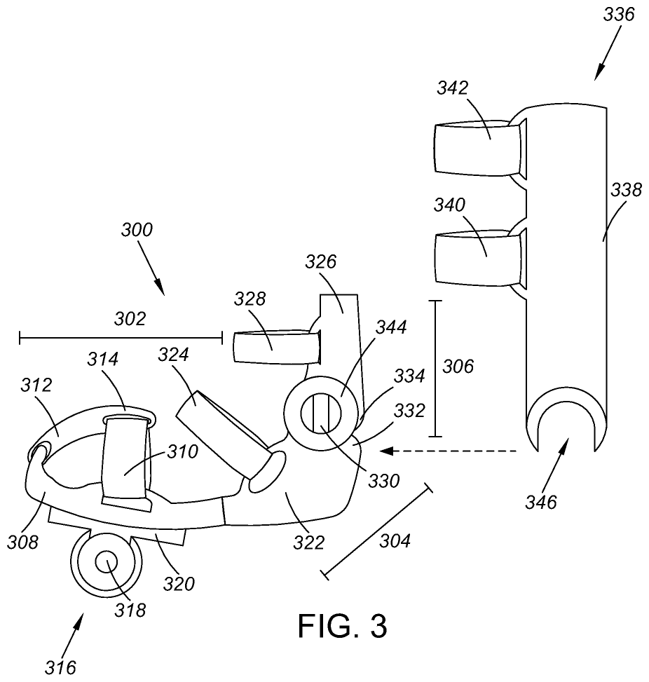

The neurological rehabilitation system can include several optional accessories that enhance the efficacy of the robotic platform. One of these accessories is the overboot. In general, a pair of detachable overboots are employed that fit over a patient's shoes and replace the previously described pedals (208 in FIGS. 2A-B). It is noted that the use of the term shoe in this description and the claims is intended to include all forms of footwear, as well as just a patient's foot in cases where the patient cannot or does not want to wear a shoe. The overboot has several advantages over the use of conventional pedals. For example, the overboot ensures that a patient's foot is secured and will not slip. In addition, the overboot is adjustable to fit the foot of any patient while keeping the ball of the patient's foot directly adjacent the distal end of the crank arm (214 in FIGS. 2A-B). Placement of the patient's foot in this manner ensures they can exert the maximum force they are capable of when pushing on the crank arm. Referring to FIG. 3, in one implementation, each overboot 300 has three sections—namely a toe section 302, a heel section 304 and a lower calf section 306. In one version, these sections 302, 304, 306 are made of nylon. The toe section 302 accommodates the front portion of a patient's foot. In the depicted version, the toe section 302 has a lower cradle section 308 that interfaces with the bottom of the patient's shoe and an open top. A transverse adjustable strap 310 is attached to opposite sides of the cradle 308 and extends over the top of a patient's shoe (not shown). The transverse strap 310 adjusts in length to that it can be cinched down and securely hold the patient's foot in place. In one version, the transverse adjustable strap 310 is a hook and loop style strap. A longitudinal strap 312 extends from the front of the cradle 308 back to the transverse strap 310 and is slidably attached to the transverse strap. For example, in the depicted version, the longitudinal strap 312 is attached to the transverse strap 310 using a loop 314. This allows the transverse strap 310 to slide through the loop 314 when it is adjusted in length so that the longitudinal strap 312 can be centered along the longitudinal midline of the patient's foot. A crank connection assembly 316 is attached to the bottom of the cradle 308. The crank connection assembly 316 includes a quick-release crank connection 318 that takes the place of the previously described pedal. The quick-release crank connection 318 releasably attaches via a quick-release mechanism to the distal end of the previously described crank arm and temporarily secures the overboot 300 (and so the patient's foot) to the crank arm during a neurological rehabilitation session. When the rehabilitation session is completed, the quick release mechanism of the quick-release crank connection 318 is employed to disconnect the overboot from the crank. Any appropriate quick release mechanism can be employed to accomplish the foregoing connection and disconnection actions. The crank connection assembly 316 also includes an adjustable base 320 disposed above the quick-release crank connection 318 that is adjustable in the longitudinal direction so that it can be moved forward or back and locked into place. In operation, once a patient's foot has been placed in the cradle 308 and the transverse strap 310 cinched down, the adjustable base 320 is moved forward or backward as needed to locate the quick-release crank connection 318 directly below the ball of the patient's foot where it is locked in place.

Referring again to FIG. 3, the heel section 304 of the overboot accommodates the rear portion of a patient's foot and is slidable attached to the proximal end of the toe section 302. More particularly, in one implementation, the heel section 304 slides in the longitudinally direction into and out of the toe section 302 so that the overall longitudinal length of the toe and heel sections can be adjusted to fit over the bottom of a patient's shoe. In the depicted version, the heel section 304 has a heel cradle section 322 that interfaces with the heel portion of the patient's shoe and has an open top. A transverse, adjustable heel strap 324 is attached to opposite sides of the heel cradle 322 and extends over the top of a patient's shoe (not shown). The transverse heel strap 324 adjusts in length so that it can be cinched down and securely hold the middle portion and heel of the patient's foot in place. In one version, the transverse heel strap 310 is a hook and loop style strap. The lower calf section 306 of the overboot accommodates the back of the lower portion of the patient's calf. In the depicted version, the lower calf section 306 has a lower calf cradle section 326 that interfaces with the back of the lower portion of the patient's calf and has an open front. A transverse, adjustable lower calf strap 328 is attached to opposite sides of the lower calf cradle 326 and extends around the patient's leg (not shown). The transverse lower calf strap 328 adjusts in length so that it can be cinched down and securely hold the patient's ankle in place during a neurological rehabilitation session. In one version, the transverse lower calf strap 328 is a hook and loop style strap. The heel section 304 and lower calf section 306 are connected together by a pair of pivotable ankle connectors 330 located on each side of the overboot 300. More particularly, the proximal end 332 of the heel cradle 322 is connected to the proximal end 334 of the lower calf cradle 326 by the pivotable ankle connectors 330. The pivotable ankle connectors 330 allow a patient to pivot their foot up and down (but not side-to-side) while pedaling during a neurological rehabilitation session. Any appropriate pivotable connector can be employed for this purpose.

The overboot 300 can also include an optional lower leg support 336 which will provide support for the patient's lower leg to keep it from bowing in or out while pedaling during a neurological rehabilitation session. The lower leg support 336 has a lower leg cradle section 338 that interfaces with the back of the patient's lower leg and an open front. A pair of transverse adjustable lower leg strap 340, 342 are attached to opposite sides of the cradle 338 and extends over the front of a patient's lower leg (not shown). One strap 340 is located about mid-way up the patient's lower leg and the other strap 342 is located near the top of the patient's lower leg (not shown). To accommodate the various lower leg lengths of patient's undergoing neurological rehabilitation, the lower leg supports 336 are made in at least three different lengths. Each lower leg strap 340, 342 adjusts in length to that it can be cinched down and securely hold the patient's lower leg in place. In one version, the lower leg straps 340, 342 are hook and loop style straps. In implementations that include the lower leg support 336, the pivotable ankle connectors 330 each include lower leg support connector 344. In one version, the lower leg support connector 344 has an extendable and retractable laterally oriented shaft with a retaining plate attached to its distal end. In this version, the lower leg support 330 has a U-shaped cutout 346 on each side of the lower leg cradle section 338 (one side of which is shown in FIG. 3). With the lower leg support connector shafts on each side of lower leg support connector 344 in their extended position, the U-shaped cutouts 346 are slid over the shafts and the shafts are retracted so that the retaining plates releasable hold the lower leg support 336 in place.

It is noted that the overboot 300 depicted in FIG. 3 is designed to fit over the right-side shoe of the patient. A left-side overboot, which is not shown, is a mirror image the right-side overboot.

### 2.2.8 Synchronized Support Rail

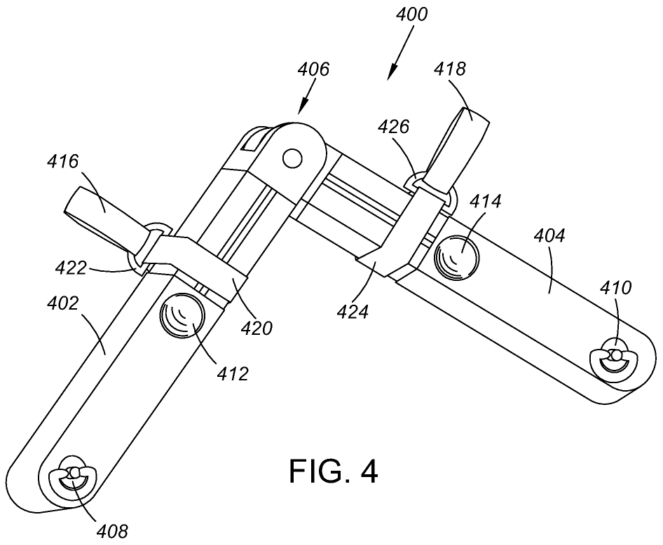

The neurological rehabilitation system can also include an optional synchronized support rail (SSR) accessory. Referring to FIG. 4, in one implementation, the SSR 400 is a rigid composite rail that has a lower rail section 402 and an upper rail section 404, which are connected together with a hinge structure 406 at their proximal ends. The hinge structure 406 allows the two rail sections 402, 404 of the SSR to pivot in a single plane from at least a 0 degree position where the SSR 400 is straight across to at least a 90 degree position where the two rail sections of the SSR are at a 90 degree angle to each other (as shown in FIG. 4). The lower rail section 402 also includes an ankle attachment 408 at its distal end. This ankle attachment 408 attaches to a mount (not shown) located on the outward facing ankle connector (330 in FIG. 3) of the overboot. The upper rail section 404 has a hip attachment 410 at its distal end. This hip attachment 410 attaches to a mount (not shown) located on the outside edge of the patient seat (206 in FIGS. 2A-B) adjacent the location where the patient's hips reside when sitting in the seat. Each of the rail sections 402, 404 is extendable and retractable, and in one version each rail section includes a locking knob 412, 414 which in a locked position locks the associated rail section at the desired length and in an unlocked position allows the associated rail to extend or retract. In this way the lower rail section 402 can be adjusted in length to accommodate the length of the patient's lower leg and the upper rail section 404 can be adjusted in length to accommodate the length of the patient's upper leg, with the hinge structure 406 aligned with the outward facing side of the patient's knee joint. Once the SSR 400 is in place and adjusted for the patient, two adjustable length straps—namely a lower rail section strap 416 and an upper rail section strap 418 are wrapped around the patients lower and upper leg, respectively, to secure the SSR to the patient's leg. In one version, the lower rail section strap 416 and the upper rail section strap 418 are hook and loop style straps. The lower rail section strap 416 is secured to the lower rail section 402 with a lower rail retaining strap 420. In the version shown in FIG. 4, the lower rail retaining strap 420 has a D-shaped ring 422 that the lower rail section strap 416 is threaded through to secure it to the lower rail retaining strap. The lower rail retaining strap 422 wraps around the lower rail section 402 but is loose enough to allow the retaining strap to slide up and down the upper part of the lower rail section. This allows the lower rail retaining strap 422 to be located at a point adjacent the patient's lower leg where it is desired to wrap the lower rail section strap 416 around the lower leg. Similarly, the upper rail section strap 418 is secured to the upper rail section 404 with an upper rail retaining strap 424. In the version shown in FIG. 4, the upper rail retaining strap 424 has a D-shaped ring 426 that the upper rail section strap 418 is threaded through to secure it to the upper rail retaining strap. The upper rail retaining strap 424 wraps around the upper rail section 404 but is loose enough to allow the retaining strap to slide up and down the lower part of the upper rail section 404. This allows the upper rail retaining strap 424 to be located at a point adjacent the patient's upper leg where it is desired to wrap the upper rail section strap 418 around the upper leg. In operation, when the SSR 400 is installed and strapped to the patient's leg as described above, it supports the patient's entire leg as they pedal the quadricycle. More particularly, the SSR 400 allows the patient to bend and straighten their leg in a plane substantially parallel to the plane in which the lower and upper rail sections pivot, while keeping their leg from bowing in or out. In this way, the patient can exert the maximum force they are capable of when pedaling. It is noted that the SSR 400 depicted in FIG. 4 is designed to strap to the outer facing side of the patient's left leg. A right-side SSR, which is not shown, is a mirror image of the depicted left-side SSR 400.

### 2.2.9 Transfer Seatbelt

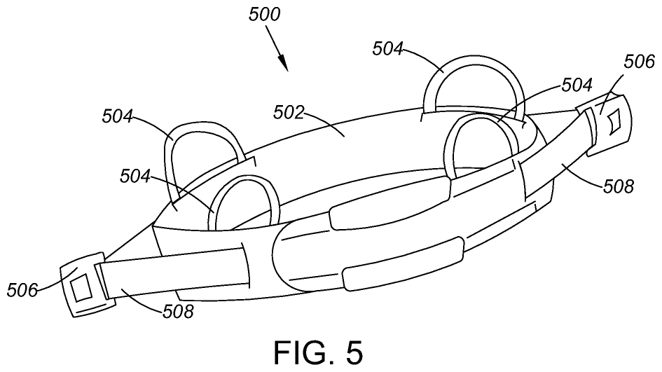

The neurological rehabilitation system can further include an optional transfer seatbelt accessory. Referring to FIG. 5, in one implementation, the transfer seatbelt 500 includes a wide, soft wraparound belt 502 that is adjustable in circumference. For example, in one version the belt 502 has overlapping ends in the front with an adjustable hook and loop style closure. The belt 502 is wrapped around the torso of the patient at about diaphragm height before mounting the robotic platform (200 in FIGS. 2A-B) and adjusted in circumference to produce a snug but comfortable fit. In one implementation, the belt 502 has four handles 504: front left, front right, rear left and rear right respectively. These handles 504 are used by medical personnel to assist with transferring a patient that is unable to mount the robotic platform on their own. In one version, each handle 504 can be stored in an adjacent pocket in the interior facing surface of the belt 502. Once the patient is seated in the robotic platform, the belt is secured on both sides to the frame of the patient seat (206 in FIGS. 2A-B). In one implementation, this is accomplish using a pair of quick-disconnect seatbelt-type latches 506 that are attached to each side of the belt 502 via a seatbelt strap 508. Each latch 506 is secured to its associated side of the patient seat using, in one version, a seatbelt-type latch plate (not shown) that is attached to the frame of the patient seat with an adjustable length strap (not shown). Once the latches 506 are connected to their associated latch plate, the adjustable latch plate straps are adjusted to secure the patient safely into the seat. In addition, the belt 502 includes at least one diaphragm sensor (not shown) that senses the expansion and contraction of the patient's diaphragm as they breath, thus providing a real-time pulmonary rate (i.e., the previously mentioned breathing rate) for the patient. This sensed breathing rate can be monitored and/or recorded during a neurological rehabilitation session. To this end, in one implementation, the seatbelt-type latches 506 and latch plate configuration includes a low-power data transfer connection that is used to transfer the diaphragm sensor signals to the neurological rehabilitation controller (102 in FIG. 1 and exemplified by 218 in FIGS. 2A-B).

### 2.3 Neurological Rehabilitation Computer Program and Adaptation Sub-Programs

Referring again to FIG. 1, the neurological rehabilitation computer program 104 employs an existing game engine 106 with integrated physics engine 108. A game engine 106 is a computer software program designed to create video games and other simulated environments, and generally includes relevant libraries and support programs. The game engine 106 typically includes a graphics rendering engine and a physics engine, and can include other support programs such as sound, scripting, animation, artificial intelligence, networking, streaming, memory management, threading, localization support, scene graph, and video support for cinematics, among others. With regard to the physics engine 108, this is a computer software program that typically provides an approximate simulation of physical systems, such as rigid body dynamics (including collision detection), soft body dynamics, and fluid dynamics. The simulations produced by the game engine are in real-time. While any suitable game engine and its integrated physics engine and other supporting programs can be employed, tested implementations of the neurological rehabilitation system used Epic Games, Inc.'s Unreal™ Engine. The particular version of the game engine employed, has been modified by various adaptation sub-programs to model and simulate human powered vehicles such as a quadricycle as it is ridden over a course.

Referring to FIGS. 1 and 2A-B, the adaptation sub-programs 110 of the neurological rehabilitation computer program 104 control the specifics and progression of the real-time visual simulation, audio and other sensory simulations, as well as the movements of the robotic platform base 202 and the resistance exhibited by the pedals 208 and steering blades 210a, 210b. These control actions are coordinated between the real-time visual simulation and the robotic platform 200 so that images seen in the real-time visualization displayed to the patient, which if they were real would affect the quadricycle and be felt by its rider (e.g., bumps or holes in the trail, the texture of the trail surface, and other physical factors), are simulated via an adaptation sub-program 110 activating the previously described articulation mechanisms 204 to induced motions of the patient portion 212 of the robotic platform 200. Thus, in general, the control actions control articulations of the patient portion 212 of the robotic platform over time in coordination with the real-time visual simulation of the task that is displayed to the patient. Another coordination example involves the tilt a quadricycle exhibits when turning. For instance, when a patient turns the simulated quadricycle during a rehabilitation session by operating the steering blades 210a, 210b, the real-time visual simulation shows the quadricycle tilt and the robotic platform base 202 tilts the patient portion 212 of the platform, in a manner that mimics what a real quadricycle would do under similar conditions in the real world. More particularly, the platform base articulates the patient portion to lean the patient in the opposite direction of the turn to simulate the lateral g-forces a rider experiences in a four-wheeled vehicle. A patient's actions during a rehabilitation session, such as the force the patient applies to the pedals 208 and steering blades 210a, 210b and the resulting speed and position of the pedals and steering blades, are seen in the real-time visual simulation as the apparent speed of the simulated quadricycle along a course and the direction the simulated quadricycle takes based on the steering blade positions.

The neurological rehabilitation computer program and its adaptation sub-programs make use of information from state modules to create the real-time-visualization and other simulations and control the robotic platform. More particularly, in one implementation depicted in FIG. 1, an environmental state module 122 and a robotic platform state module 124 are employed. The environmental state module 122 provides various environmental states that inform the neurological rehabilitation computer program 104 and its adaptation sub-programs 110 of simulated environmental conditions applicable to the simulated task over time (e.g., the environmental conditions change as the simulated task progresses). In general, the types of environmental conditions that are stored in the environmental state library 122 are those that produce simulated external forces that effect the simulated quadricycle and its rider. Thus, these environmental states are employed in the generation of the real-time visual simulation as well as in the control of the robotic platform 114. For example, some of the environmental conditions include the current wind speed and direction, the current incline (upwards or downwards) of the trail, the current texture of the surface of the trail (e.g., rough, smooth, paved, dirt, gravel, and so on). The values assigned to these types of environmental conditions over the course of the simulation can be defined ahead of time and stored in the environmental state module 122. It is noted that more than one set of environmental conditions can be created and stored for a simulation. Thus, a set of environmental conditions can be selected prior to initiating the simulation to tailor a rehabilitation session to the abilities of a patient. For example, a set of mild environmental conditions can be chosen for a patient that is new to the neurological rehabilitation process, whereas a set of more extreme conditions could be selected for a patient that has logged several neurological rehabilitation sessions.

The robotic platform state module 124 collects on an ongoing basis the information needed by the game engine and adaptation subprograms to simulate the quadricycle ride in the next time step of the simulation. For example, this information includes the current conditions of the various components of the robotic platform 114, such as the robotic platform orientation, the resistance values of the servo motors associated with the pedals and/or steering blades, the force the patient's feet and/or hands are applying to the pedals/steering blades, the rotational speed and position of the servo motors, and so on. In one implementation, this information is the previously described processed outputs of the sensor array and is obtained from the neurological rehabilitation controller.

### 2.3.1 Human-Powered Vehicle Real-Time Visual Simulation Adaptation Sub-Program

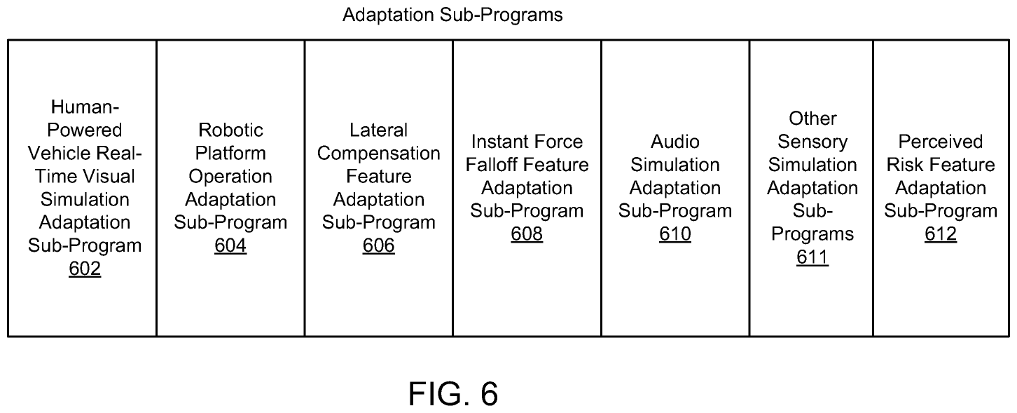

Referring to FIG. 6, in one implementation, a human-powered vehicle real-time visual simulation adaptation sub-program 602 is employed to supplement the game engine's simulation. For example, in the case of a quadricycle simulation, the human-powered vehicle real-time visual simulation adaptation sub-program 602 provides the additional physics computations needed to simulate aspects of the overall real-time visual simulation that are unique to a human-powered vehicle (e.g., a quadricycle) and not available in the game engine. Thus, for example, the game engine along with the human-powered vehicle real-time visual simulation adaptation sub-program 602 can produce a real-time visual simulation showing the quadricycle (or at least the front part of the quadricycle) as it moves along a course such as a bike trail in the woods.

For example, in one implementation, the human-powered vehicle real-time visual simulation adaptation sub-program 602 causes the quadricycle simulation to change (e.g., the speed of the quadricycle along the trail) based in part on the amount of force being applied by the patient's left and right feet to the left and right foot interfaces, and/or the amount of force being applied by the patient's left and right hands to the left and right hand interfaces.

### 2.3.2 Robotic Platform Operation Adaptation Sub-Program

Referring again to FIG. 6, in one implementation, a robotic platform operation adaptation sub-program 604 is employed to generate control instructions to orient the patient portion of the robotic platform and to control the amount of resistance that is exhibited by the servo motors associated with the pedals and/or steering blades on an ongoing basis. The robotic platform operation adaptation sub-program 404 accesses the robotic platform state module to obtain information about the current conditions of the various components of the robotic platform that is needed to simulate the quadricycle ride in the next time step of the simulation. Examples of some of these control instructions are described in the paragraphs and sections to follow. It is noted that the robotic platform operation adaptation sub-program 604 is in communication with and coordinates with the human-powered vehicle real-time visual simulation adaptation sub-program 602 to synchronize the changes to the robotic platform to the real-time visual simulation.

With regard to the resistance applied to the pedals and steering blades by the servo motors, the robotic platform operation adaptation sub-program 604 dynamically controls the level of the resistance during a rehabilitation session based on both the simulated quadricycle ride and the capabilities of the patient. More particularly, the resistance level applied based on the simulated quadricycle ride reflects simulated external factors. For example, if the simulation involves riding the simulated quadricycle up a hill, the resistance applied to the pedals would be increased appropriately to mimic a rider having to increase their pedaling force to move the quadricycle up the hill, albeit limited by the patient factors to be described next. Conversely, if the simulation involves riding the simulated quadricycle down a hill, the resistance applied to the pedals would be decreased appropriately to mimic the reduction in the pedaling force required to move the quadricycle down the hill. In general, any simulated external factor (e.g., inertia and momentum from built up virtual speed that in some circumstances would reduce the amount of force that needs to be applied to the pedals) that if real would affect the amount of force a rider would have to exert to pedal a quadricycle, is reflected in an appropriate increase or decrease in the resistance applied to the pedals. In one implementation, a similar resistance scheme is employed for the steering blades.

The resistance levels applied to the pedals and steering blades during a rehabilitation session can also be based on the capabilities of the patient. More particularly, in one implementation, the foregoing involves monitoring the force a patient is applying with each limb using the previously described force sensors and limiting resistance applied to the pedals and steering blades so that the patient does not apply a force with their arms or legs that exceeds a prescribed maximum. For example, a rehabilitation therapist or medical professional could set the maximum force he or she deems safe for a patient to apply with their legs and/or arms.

### 2.3.3 Lateral Compensation

Referring again to FIG. 6, in one implementation, a lateral compensation feature adaptation sub-program 606 is employed. Lateral compensation uses the input from sensors associated with the pedals and/or the steering blades to identify a lateral limb strength imbalance. For example, if one arm is not as strong as the other or one leg is not as strong as the other, a lateral limb strength imbalance exists. This imbalance can be the result of various neurological or physical impairments. For instance, a stroke victim often experiences impairment of a limb or limbs on one side. Another example is an amputee with a prosthetic limb, where the remaining limb is stronger than the side with the prosthesis.

In one implementation, a neurological rehabilitation therapist or medical professional manually sets the level of reduced resistance that is applied for an impaired limb using the system monitor (which will be described in greater detail shortly) to approximately equalize the locomotive effect of the patient's pedaling. In one implementation, the lateral compensation for a patient's legs relies on detecting when the patient's impaired leg is pushing the associated pedal. This is possible by monitoring the rotational position of the pedals as the impaired leg will begin pushing the associated pedal at a point where the un-impaired leg is fully extended. In one implementation, the lateral compensation for a patient's arms assumes that the patient primarily pulls down on a steering blade with one arm to initiate a turn or otherwise guide the virtual quadricycle, while the other arm simply assists by pushing up with less force on the other steering blade. Given this assumption, the rotational position sensor(s) associated with the steering blade servo motor can be monitored to determine when a patient's impaired arm is pulling the associated steering blade based on the direction of rotation. This sensor monitoring and the reduced resistance level setting are used to reduce the resistance applied by the pedal servo motor on the pedals and/or the resistance applied by the steering servo motor on the steering blades, when the impaired limb is pushing or pulling, by the set amount. In this way, the lateral limb strength imbalance is compensated for so that the patient “feels” like they are pedaling or moving the pedals and/or steering blades with equal force. It is noted that the controller ignores the reduction in the resistance applied to the pedals and/or steering blades when an impaired limb is pushing or pulling and generates the real-time visual simulation as if the impaired limb was pushing or pulling with the same force as the un-impaired limb.

In another implementation, the force exerted by the stronger of the patient's two legs and/or the stronger of the patient's two arms is measured continuously using the previously described force sensors. The measured force is deemed to be the intended force that the rider wants to exert with both arms and/or both legs. The difference between the intended force and the actual force being exerted by the impaired limb is calculated. Based on this force difference, the resistance applied by the pedal servo motor on the pedal and/or the resistance applied by the steering servo motor on the steering blade, when the impaired limb is pushing or pulling (as detected in the manner described previously) is reduced by an amount that equalizes the locomotive effect of the rider's pedaling or steering blade movements. In this way, the lateral limb strength imbalance is compensated for so that the patient “feels” like they are moving the pedals and/or steering blades with equal force. Here again, the controller ignores the reduction in the resistance applied to the pedals and/or steering blades when an impaired limb is pushing or pulling and generates the real-time visual simulation as if the impaired limb was pushing or pulling with the same force as the un-impaired limb.

In implementations incorporating both the lateral compensation feature and the previously described maximum force feature, the maximum force limit would take precedence over a resistance computed for that limb based on the lateral compensation feature. In other words, if the force required for a patient to move a pedal or steering blade given a resistance computed based on the lateral compensation feature exceeds the maximum force limit set for that limb, the resistance is reduced so as to not exceed the maximum force limit. On the other hand, if the force required for a patient to move a pedal or steering blade given a resistance computed based on the lateral compensation feature is less than the maximum force limit set for that limb, the resistance computed using the lateral compensation feature is employed.

### 2.3.4 Instant Force Falloff

Referring to FIG. 6 once again, in one implementation, an instant force falloff (IFF) feature adaptation sub-program 608 is employed. The IFF provides a more realistic “biking” experience by simulating a coasting mode. When the patient stops pedaling, without the IFF the resistance on the pedals created by the servo motors would cause the pedals to move “backwards” (i.e., the opposite direction from the pedaling) to create a bounce-like effect caused by a latency in the servo motors response to a stopped pedaling event. This effect does not exist in the real world. Coast mode is activated as appropriate to the circumstances to prevent the bounce-like effect.

IFF is initiated when pedal sensors sense a pedal moving in the “backwards” direction or not moving at all. When such a movement is sensed, the resistance force placed on the pedals by the motors is ceased. The servo motors are operated in torque mode to provide a low-latency response along with very fast control board operating speeds (e.g., 120 MHz receiving/sending instructions). This process happens so fast that no “bounce” is felt by the rider. In one implementation, a similar IFF scheme is employed for the steering blades.

### 2.3.5 Audio and Other Sensory Simulation Adaptation Sub-Programs

Referring to FIG. 6, in one implementation, an audio simulation adaption sub-program 610 is employed. The audio simulation adaption sub-program 610 generates an audio output 119 that is synchronized with the real-time visual simulation described previously. The audio output 119 would be played to the patient during the rehabilitation session along with the real-time visual simulation. As described previously, the audio would mimic the ambient sounds that would typically be heard by a quadricycle rider riding along a real course.

Referring again to FIG. 6, in one implementation, other sensory simulation adaptation sub-programs 611 are employed. For example, a sensory simulation adaptation sub-program 611 for providing instructions to a scent producing component could be incorporated to mimic the environmental smells a patient would experience during a real quadricycle ride. This scent producing simulation sub-program generates instructions that are synchronized with the real-time visual simulation described previously. The instructions are output to scent producing components that would produce various scents during the rehabilitation session along with the real-time visual simulation.

### 2.3.6 Enhanced Task-Oriented Therapy

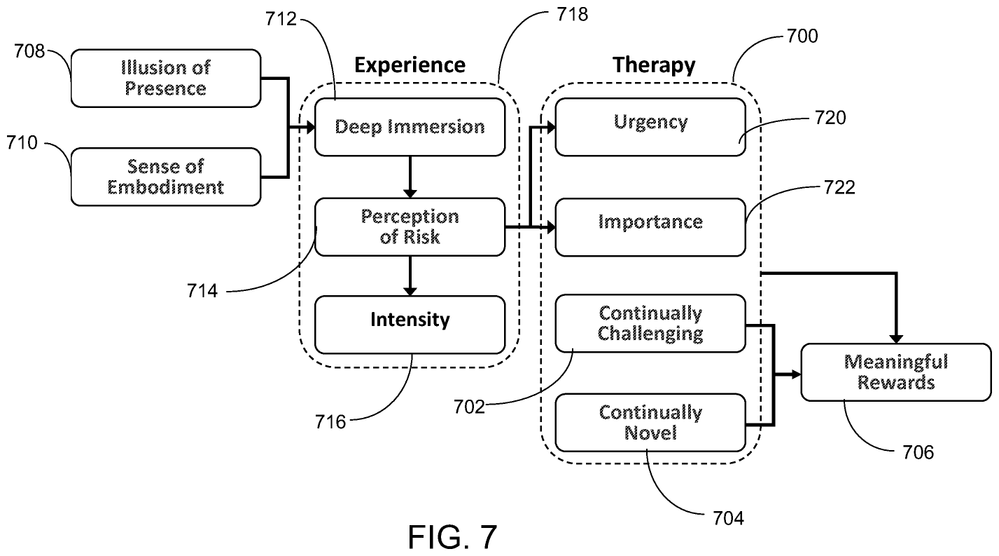

As described previously, and referring to FIG. 7, it is believed that enhanced task-oriented therapy 700 utilizing virtual reality and robotic assistance to simulate tasks that are continually challenging 702 and continually novel 704 with meaningful rewards 706 (e.g., satisfactorily completing a difficult task, “beating the game”, and so on) results in more successful neurological rehabilitation treatments. The neurological rehabilitation system implementations described herein achieve this goal by delivering the illusion of presence 708 and sense of embodiment 710 in a task required for a patient to achieve deep immersion 712. This deep immersion in a task allows a patient to suspend their disbelief that they are simply in a simulation, which then allows a perception of risk 714 to be injected into the simulated task. This, in turn, creates two important capabilities. First, it adds intensity 716 to the overall experience 718 which unto itself can improve neuroplasticity but perhaps more importantly, it allows using the perception of risk to assign urgency 720 and importance 722 to the actions performed during the enhanced task-oriented therapy 700.

The neurological rehabilitation system implementations described herein have the unique ability to introduce a perceived risk in a task-oriented therapy session. Generally, as indicated above, a perceived risk in task-oriented therapy involves presenting a patient with a constant flow of new challenges that require the patient to physically react. For example, referring again to FIG. 6, in the context of the neurological rehabilitation systems implementations described herein, a perceived risk feature adaptation sub-program 612 is employed. This sub-program 612 introduces obstacles into the quadricycle real-time visual simulation that a patient must deal with over the course of a session. For example, a rock or other object can appear on the trail ahead which requires the rider to reduce their apparent speed by pedaling slower and using the steering blades to steer around the obstacle. The same is true of a sudden curve in the trail or encountering a narrow bridge. Each of these requires the patient to react with a mental urgency and importance because it is perceived as an imminent risk.

### 2.4 System Monitor

As indicated previously, in one implementation the neurological rehabilitation system includes a system monitor (124 in FIG. 1) that is in two-way communication with the neurological rehabilitation controller. In one version, the system monitor is a computer workstation that includes at least one display and various user input devices. This workstation 218 can be incorporated into the robotic platform 200 as shown in FIGS. 2A-B, or it can be a stand-alone device located remotely form the robotic platform. In this later implementation, the system monitor is in two-way communication with the neurological rehabilitation controller via a wired connection if it is nearby but can also communicate via a wireless connection either locally (using RF or IR or a local intranet) or from anywhere using a computer network connection such as the Internet. In general, the system monitor receives information about the neurological rehabilitation system and rehabilitation sessions from the neurological rehabilitation controller via the neurological rehabilitation computer program. The system monitor can also be employed to input data and commands, to set parameters, make changes and add new features to the neurological rehabilitation computer program. This can include making changes and/or adding new data to the environmental state and robotic platform state modules.

The system monitor allows an operator such as a neurological rehabilitation therapist or medical professional to observe the real-time visual simulation that the patient sees on the monitor's display. In addition, readouts from the various sensors can be displayed to the operator. For example, the current resistance values of the servo motors associated with the pedals and/or steering blades, the current force the patient's feet and/or hands are applying to the pedals/steering blades, the rotational speed and position of the servo motors, and so on can be displayed. Further, the current robotic platform orientation can be displayed to the operator.

### 2.5 Other Advantages and Implementations

While the neurological rehabilitation system has been described by specific reference to implementations thereof, it is understood that variations and modifications thereof can be made without departing from the true spirit and scope of the system. By way of example but not limitation, while the neurological rehabilitation system described so far involved simulating a quadricycle for use in neurological rehabilitation task-oriented therapy, the system can take other forms. In general, any form that requires a patient to use their limbs (e.g., arms, legs, or both) to move human-machine interfaces of a robotic platform in a way that results in synchronized simulated locomotion in a real-time visual simulation of a task being displayed to the patient, can be employed. Other possible forms might be a robotic platform and real-time visual simulation that mimics a person skiing, or walking, or hiking, or participating in a sporting event, to name just a few examples. Further, while the system described so far is employed for neurological rehabilitation, this need not be the case. Rather, the system can be employed for physical rehabilitation therapy, or general exercise, or e-sports, to name a few alternate uses. Thus, in general, the system could be referred to as a training system that could be used for neurological rehabilitation task-oriented therapy.

Such a training system for a human subject would include a visual display component having a display, and a robotic platform including a lower body apparatus that is contacted by a subject's left foot and right foot and that presents a resistive force against which the subject uses lower body muscles to move the lower body apparatus with the subject's left foot and right foot. The training system could optionally also include an upper body apparatus that is contacted by the subject's left and right hands and that presents a resistive force which requires the subject to use upper body muscles to move the upper body apparatus with the subject's left and right hands. The training system further includes a sensor array including at least one left leg sensor that measures the amount of force being applied by the subject's left leg to the lower body apparatus and at least one right leg sensor that measures the amount of force being applied by the subject's right leg to the lower body apparatus over time. In the case where the training system includes an upper body apparatus, the sensor array also includes at least one left arm sensor that measures the amount of force being applied by the subject's left arm to the upper body apparatus and at least one right arm sensor that measures the amount of force being applied by the subject's right arm to the upper body apparatus over time. A training controller having one or more computing devices, and a training computer program having a plurality of sub-programs executable by the computing device or devices is also included in the training system. The sub-programs configure the computing device or devices to control the amount of resistive force applied by the lower body apparatus to a left foot interface with the subject's left foot over time during a training session of the subject and control the amount of resistive force applied by the lower body apparatus to a right foot interface with the subject's right foot over time during the training session of the subject. In addition, the sub-programs generate a real-time visual simulation of a training task that is displayed to the subject via the visual component display over time during the training session of the subject based in part on the amount of force being applied by the subject's left foot to the left foot interface and right foot to the right foot interface. In the case where the training system includes an upper body apparatus, the sub-programs also configure the computing device or devices to control the amount of resistive force applied by the upper body apparatus to a left hand interface with the subject's left hand over time during the training session of the subject and control the amount of resistive force applied by the upper body apparatus to a right hand interface with the subject's right hand over time during the training session of the subject. In addition, the sub-programs also generate a real-time visual simulation of a task that is displayed to the subject via the visual component display over time during the training session of the subject based in part on the amount of force being applied by the subject's left and right hands to the left and right hand interfaces.

Further, while the steering blades described previously employed a single steering blade servo motor, in an alternate implementation, the steering blades employed on the neurological rehabilitation robotic platform are cantilevered beams having an adjustable length and hand grips located at their distal ends, as before. However, in this implementation, the proximal end of each steering blade is attached to a different steering blade servo motor. The steering blades are still operated by moving them in a vertical up-down motion. However, in this implementation, each servo motor attached to a steering blade allows the blade to be moved up and down resulting in a clockwise or counter-clockwise rotation of the server motor axis. In the case of the steering blade located on the right side of the patient when sitting in the patient seat, an upward motion results in a counterclockwise rotation of the servo motor axis and a downward motion results in a clockwise rotation. In the case of the steering blade located on the left side of the patient when sitting in the patient seat, an upward motion results in a clockwise rotation of the servo motor axis and a downward motion results in a counter clockwise rotation. The steering blades again extend forward from their attachment to the servo motors and in a neutral position (i.e., the position where the blades are not turning the virtual quadricycle to the left or right) are horizontally level. In one version, an upward motion of steering blade located on the left side of the patient and corresponding downward motion of the steering blade located on the right side of the patient, simulates a right-hand turn. Conversely, an upward motion of steering blade located on the right side of the patient and corresponding downward motion of the steering blade located on the left side of the patient, simulates a left-hand turn. The extent of the up-down motion of the blades dictates the degree of the simulated turn. This up-down motion is designed to engage a patient's upper body skeletal-muscular system to steer the virtual quadricycle during a neurological rehabilitation session and is thought to enhance and increase neuroplasticity. The servo motor attached to each steering blade is controlled by the neurological rehabilitation controller and used to create a resistance force on the blades that the patient must overcome to move the blade.

It is further noted that any or all of the implementations that are described in the present document and any or all of the implementations that are illustrated in the accompanying drawings may be used and thus claimed in any combination desired to form additional hybrid implementations. In addition, although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What has been described above includes example implementations. It is, of course, not possible to describe every conceivable combination of components or methodologies for purposes of describing the claimed subject matter, but one of ordinary skill in the art may recognize that many further combinations and permutations are possible. Accordingly, the claimed subject matter is intended to embrace all such alterations, modifications, and variations that fall within the spirit and scope of the appended claims.

In regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the foregoing implementations include a system as well as a computer-readable storage media having computer-executable instructions for performing the acts and/or events of the various methods of the claimed subject matter.

There are multiple ways of realizing the foregoing implementations (such as an appropriate application programming interface (API), tool kit, driver code, operating system, control, standalone or downloadable software object, or the like), which enable applications and services to use the implementations described herein. The claimed subject matter contemplates this use from the standpoint of an API (or other software object), as well as from the standpoint of a software or hardware object that operates according to the implementations set forth herein. Thus, various implementations described herein may have aspects that are wholly in hardware, or partly in hardware and partly in software, or wholly in software.

The aforementioned systems have been described with respect to interaction between several components. It will be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (e.g., hierarchical components).

Additionally, it is noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

### 3.0 Exemplary Operating Environments

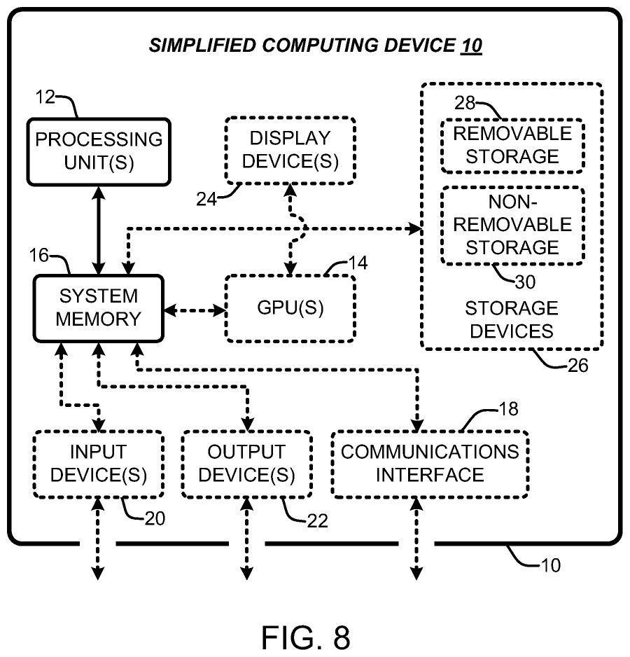

The neurological rehabilitation system implementations described herein are operational within numerous types of general purpose or special purpose computing system environments or configurations. FIG. 8 illustrates a simplified example of a general-purpose computer system on which various implementations and elements of the neurological rehabilitation system, as described herein, may be implemented. It is noted that any boxes that are represented by broken or dashed lines in the simplified computing device 10 shown in FIG. 8 represent alternate implementations of the simplified computing device. As described below, any or all of these alternate implementations may be used in combination with other alternate implementations that are described throughout this document. The simplified computing device 10 is typically found in devices having at least some minimum computational capability such as personal computers (PCs), server computers, handheld computing devices, laptop or mobile computers, communications devices such as cell phones and personal digital assistants (PDAs), multiprocessor systems, microprocessor-based systems, set top boxes, programmable consumer electronics, network PCs, minicomputers, mainframe computers, and audio or video media players.

To allow a device to realize the neurological rehabilitation system implementations described herein, the device should have a sufficient computational capability and system memory to enable basic computational operations. In particular, the computational capability of the simplified computing device 10 shown in FIG. 8 is generally illustrated by one or more processing unit(s) 12, and may also include one or more graphics processing units (GPUs) 14, either or both in communication with system memory 16. Note that that the processing unit(s) 12 of the simplified computing device 10 may be specialized microprocessors (such as a digital signal processor (DSP), a very long instruction word (VLIW) processor, a field-programmable gate array (FPGA), or other micro-controller) or can be conventional central processing units (CPUs) having one or more processing cores.

In addition, the simplified computing device 10 may also include other components, such as, for example, a communications interface 18. The simplified computing device 10 may also include one or more conventional computer input devices 20 (e.g., touchscreens, touch-sensitive surfaces, pointing devices, keyboards, audio input devices, voice or speech-based input and control devices, video input devices, haptic input devices, devices for receiving wired or wireless data transmissions, and the like) or any combination of such devices.

Similarly, various interactions with the simplified computing device 10 and with any other component or feature of the neurological rehabilitation system implementations described herein, including input, output, control, feedback, and response to one or more users or other devices or systems associated with the neurological rehabilitation system implementations, are enabled by a variety of Natural User Interface (NUI) scenarios. The NUI techniques and scenarios enabled by the TDR matrix suction sensor implementations include, but are not limited to, interface technologies that allow one or more users to interact with the TDR matrix suction sensor implementations in a “natural” manner, free from artificial constraints imposed by input devices such as mice, keyboards, remote controls, and the like.

Such NUI implementations are enabled by the use of various techniques including, but not limited to, using NUI information derived from user speech or vocalizations captured via microphones or other sensors (e.g., speech and/or voice recognition). Such NUI implementations are also enabled by the use of various techniques including, but not limited to, information derived from a user's facial expressions and from the positions, motions, or orientations of a user's hands, fingers, wrists, arms, legs, body, head, eyes, and the like, where such information may be captured using various types of 2D or depth imaging devices such as stereoscopic or time-of-flight camera systems, infrared camera systems, RGB (red, green and blue) camera systems, and the like, or any combination of such devices. Further examples of such NUI implementations include, but are not limited to, NUI information derived from touch and stylus recognition, gesture recognition (both onscreen and adjacent to the screen or display surface), air or contact-based gestures, user touch (on various surfaces, objects or other users), hover-based inputs or actions, and the like. Such NUI implementations may also include, but are not limited, the use of various predictive machine intelligence processes that evaluate current or past user behaviors, inputs, actions, etc., either alone or in combination with other NUI information, to predict information such as user intentions, desires, and/or goals. Regardless of the type or source of the NUI-based information, such information may then be used to initiate, terminate, or otherwise control or interact with one or more inputs, outputs, actions, or functional features of the neurological rehabilitation system implementations described herein.

However, it should be understood that the aforementioned exemplary NUI scenarios may be further augmented by combining the use of artificial constraints or additional signals with any combination of NUI inputs. Such artificial constraints or additional signals may be imposed or generated by input devices such as mice, keyboards, and remote controls, or by a variety of remote or user worn devices such as accelerometers, electromyography (EMG) sensors for receiving myoelectric signals representative of electrical signals generated by user's muscles, heart-rate monitors, galvanic skin conduction sensors for measuring user perspiration, wearable or remote biosensors for measuring or otherwise sensing user brain activity or electric fields, wearable or remote biosensors for measuring user body temperature changes or differentials, and the like. Any such information derived from these types of artificial constraints or additional signals may be combined with any one or more NUI inputs to initiate, terminate, or otherwise control or interact with one or more inputs, outputs, actions, or functional features of the neurological rehabilitation system implementations described herein.

The simplified computing device 10 may also include other optional components such as one or more conventional computer output devices 22 (e.g., display device(s) 24, audio output devices, video output devices, devices for transmitting wired or wireless data transmissions, and the like). Note that typical communications interfaces 18, input devices 20, output devices 22, and storage devices 26 for general-purpose computers are well known to those skilled in the art, and will not be described in detail herein.

The simplified computing device 10 shown in FIG. 8 may also include a variety of computer-readable media. Computer-readable media can be any available media that can be accessed by the computer 10 via storage devices 26, and can include both volatile and nonvolatile media that is either removable 28 and/or non-removable 30, for storage of information such as computer-readable or computer-executable instructions, data structures, programs, sub-programs, or other data. Computer-readable media includes computer storage media and communication media. Computer storage media refers to tangible computer-readable or machine-readable media or storage devices such as digital versatile disks (DVDs), blu-ray discs (BD), compact discs (CDs), floppy disks, tape drives, hard drives, optical drives, solid state memory devices, random access memory (RAM), read-only memory (ROM), electrically erasable programmable read-only memory (EEPROM), CD-ROM or other optical disk storage, smart cards, flash memory (e.g., card, stick, and key drive), magnetic cassettes, magnetic tapes, magnetic disk storage, magnetic strips, or other magnetic storage devices. Further, a propagated signal is not included within the scope of computer-readable storage media.

Retention of information such as computer-readable or computer-executable instructions, data structures, programs, sub-programs, and the like, can also be accomplished by using any of a variety of the aforementioned communication media (as opposed to computer storage media) to encode one or more modulated data signals or carrier waves, or other transport mechanisms or communications protocols, and can include any wired or wireless information delivery mechanism. Note that the terms “modulated data signal” or “carrier wave” generally refer to a signal that has one or more of its characteristics set or changed in such a manner as to encode information in the signal. For example, communication media can include wired media such as a wired network or direct-wired connection carrying one or more modulated data signals, and wireless media such as acoustic, radio frequency (RF), infrared, laser, and other wireless media for transmitting and/or receiving one or more modulated data signals or carrier waves.

Furthermore, software, programs, sub-programs, and/or computer program products embodying some or all of the various neurological rehabilitation system implementations described herein, or portions thereof, may be stored, received, transmitted, or read from any desired combination of computer-readable or machine-readable media or storage devices and communication media in the form of computer-executable instructions or other data structures. Additionally, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, or media.

The neurological rehabilitation system implementations described herein may be further described in the general context of computer-executable instructions, such as programs, sub-programs, being executed by a computing device. Generally, sub-programs include routines, programs, objects, components, data structures, and the like, that perform particular tasks or implement particular abstract data types. The neurological rehabilitation system implementations may also be practiced in distributed computing environments where tasks are performed by one or more remote processing devices, or within a cloud of one or more devices, that are linked through one or more communications networks. In a distributed computing environment, sub-programs may be located in both local and remote computer storage media including media storage devices. Additionally, the aforementioned instructions may be implemented, in part or in whole, as hardware logic circuits, which may or may not include a processor. Still further, the neurological rehabilitation system implementations described herein can be virtualized and realized as a virtual machine running on a computing device such as any of those described previously. In addition, multiple virtual machines can operate independently on the same computer device.

Alternatively, or in addition, the functionality described herein can be performed, at least in part, by one or more hardware logic components. For example, and without limitation, illustrative types of hardware logic components that can be used include FPGAs, application-specific integrated circuits (ASICs), application-specific standard products (ASSPs), system-on-a-chip systems (SOCs), complex programmable logic devices (CPLDs), and so on.

Wherefore, what is claimed is:

1. A training system for a human subject, comprising:
a visual display component;
a robotic platform comprising a lower body apparatus that is contacted by a subject's left foot and right foot and that presents a resistive force which requires the subject to use lower body muscles to move the lower body apparatus with the subject's left foot and right foot;
a sensor array comprising at least one left leg sensor that measures the amount of force being applied by the subject's left leg to the lower body apparatus and at least one right leg sensor that measures the amount of force being applied by the subject's right leg to the lower body apparatus over time; and
a training controller comprising one or more computing devices, and a training computer program having a plurality of sub-programs executable by said computing device or devices, wherein the sub-programs configure said computing device or devices to,
control the amount of resistive force applied by the lower body apparatus to a left foot interface with the subject's left foot over time during a training session of the subject and control the amount of resistive force applied by the lower body apparatus to a right foot interface with the subject's right foot over time during the training session of the subject, and
generate a real-time visual simulation of a training task that is displayed to the subject via the visual display component over time during the training session of the subject based in part on the amount of force being applied by the subject's left foot to the left foot interface and right foot to the right foot interface.

2. The training system of claim 1, wherein:
the robotic platform further comprises an upper body apparatus that is contacted by the subject's left and right hands and that presents a resistive force which requires the subject to use upper body muscles to move the upper body apparatus with the subject's left and right hands;
the sensor array further comprises at least one left arm sensor that measures the amount of force being applied by the subject's left arm to the upper body apparatus and at least one right arm sensor that measures the amount of force being applied by the subject's right arm to the upper body apparatus over time; and
the training computer program further comprises sub-programs which configure said computing device or devices to,
control the amount of resistive force applied by the upper body apparatus to a left hand interface with the subject's left hand over time during the training session of the subject and control the amount of resistive force applied by the upper body apparatus to a right hand interface with the subject's right hand over time during the training session of the subject, and
generate a real-time visual simulation of a task that is displayed to the subject via the visual display component over time during the training session of the subject based in part on the amount of force being applied by the subject's left and right hands to the left and right hand interfaces.

3. A neurological rehabilitation system for treatment of nervous system injuries and neurological diseases, comprising:
a visual display component;
a robotic platform comprising a lower body apparatus that is contacted by a patient's left foot and right foot and that presents a resistive force which requires the patient to use lower body muscles to move the lower body apparatus with the patient's left foot and right foot;
a sensor array comprising at least one left leg sensor that measures the amount of force being applied by the patient's left leg to the lower body apparatus and at least one right leg sensor that measures the amount of force being applied by the patient's right leg to the lower body apparatus over time; and
a neurological rehabilitation controller comprising one or more computing devices, and a neurological rehabilitation computer program having a plurality of sub-programs executable by said computing device or devices, wherein the sub-programs configure said computing device or devices to,
control the amount of resistive force applied by the lower body apparatus to a left foot interface with the patient's left foot over time during a neurological rehabilitation session of the patient and control the amount of resistive force applied by the lower body apparatus to a right foot interface with the patient's right foot over time during the neurological rehabilitation session of the patient, and
generate a real-time visual simulation of a task that is displayed to the patient via the visual display component over time during the neurological rehabilitation session of the patient based in part on the amount of force being applied by the patient's left foot to the left foot interface and right foot to the right foot interface.

4. The neurological rehabilitation system of claim 3, wherein the robotic platform takes the form of a stationary human-powered recumbent quadricycle, the left foot interface takes the form of a left-side pedal, and the right foot interface takes the form of a right-side pedal, and wherein the resistive force applied to the left-side pedal and the right-side pedal is applied by a pedal servo motor of the lower body apparatus.

5. The neurological rehabilitation system of claim 3, wherein:
the sensor array further comprises a left-side pedal sensor that measures the movement of the left-side pedal and a right-side pedal sensor that measures the movement of the right-side pedal; and wherein
the neurological rehabilitation computer program further comprises coasting mode sub-programs that configure said computing device or devices to further control the amount of resistive force applied by the lower body apparatus to the left and right foot interfaces to provide an instant force falloff to simulate a coasting mode when the patient stops pedaling under prescribed circumstances, said coasting mode sub-programs configuring said computing device or devices to:
periodically using the left-side and right-side pedal sensors to detect if the left-side and right-side pedals have stopped moving or have started moving in a direction opposite the direction the pedals were moving the immediately previous time the left-side and right-side pedal sensors were used to detect movement of the left-side and right-side pedals; and
whenever it is detected that the left-side and right-side pedals have stopped moving or have started moving in the opposite direction, the resistive force applied to the left-side pedal and the right-side pedal by the pedal servo motor of the lower body apparatus is changed to zero.

6. The neurological rehabilitation system of claim 3, wherein:
the robotic platform further comprises an upper body apparatus that is contacted by a patient's left and right hands and that presents a resistive force which requires the patient to use upper body muscles to move the upper body apparatus with the patient's left and right hands;
the sensor array further comprises at least one left arm sensor that measures the amount of force being applied by the patient's left arm to the upper body apparatus and at least one right arm sensor that measures the amount of force being applied by the patient's right arm to the lower body apparatus over time; and
the neurological rehabilitation computer program further comprises sub-programs which configure said computing device or devices to,
control the amount of resistive force applied by the upper body apparatus to a left hand interface with the patient's left hand over time during the neurological rehabilitation session of the patient and a right hand interface with the patient's right hand over time during the neurological rehabilitation session of the patient, and
generate a real-time visual simulation of a task that is displayed to the patient via the visual display component over time during the neurological rehabilitation session of the patient based in part on the amount of force being applied by the patient's left and right hands to the left and right hand interfaces.

7. The neurological rehabilitation system of claim 6, wherein the robotic platform takes the form of a stationary human-powered recumbent quadricycle, the left foot interface takes the form of left-side pedal, and the right foot interface takes the form of right-side pedal, the left hand interface takes the form of left-side steering blade, and the right hand interface takes the form of right-side steering blade, and wherein the resistive force applied to the left-side and right-side pedals is applied by a pedal servo motor of the lower body apparatus, and the resistive force applied to the left-side and right-side steering blade is applied by a steering blade servo motor of the upper body apparatus.

8. The neurological rehabilitation system of claim 7, wherein the left-side and right-side steering blades each comprise an extendable and retractable cantilevered beam, which is attached at a distal end to a steering blade axle mechanism that is attached to the steering blade servo motor, and which comprises a hand grip at a distal end, each of said steering blades being operated by moving the steering blade in an up-down motion, and wherein the left-side and right-side steering blades are interlocked laterally such that when one steering blade is moved downward, the other moves upward, and wherein moving a first one of the steering blades upward and simultaneously moving the other steering blade downward simulates a turn of the stationary human-powered recumbent bike in a first direction, and wherein moving the first one of the steering blades downward and simultaneously moving the other steering blade upward simulates a turn of the stationary human-powered recumbent bike in a second direction.

9. The neurological rehabilitation system of claim 6, wherein the neurological rehabilitation computer program further comprises sub-programs that configure said computing device or devices to further control the amount of resistive force applied by the upper body apparatus to the left and right hand interfaces to provide lateral compensation for an imbalance in lateral arm strength.

10. The neurological rehabilitation system of claim 3, wherein:
the robotic platform further comprises a patient portion that accommodates the patient and a base attached to the patient portion which comprises at least one articulation apparatus that articulates the patient portion of the robotic platform to produce various pitch or roll conditions, or combinations thereof, and wherein
the neurological rehabilitation computer program further comprises a sub-program that configures said computing device or devices to,
control articulations of the patient portion of the robotic platform over time in synchronization with the real-time visual simulation of the task that is displayed to the patient via the visual display component.

11. The neurological rehabilitation system of claim 3, wherein the neurological rehabilitation computer program further comprises sub-programs that configure said computing device or devices to further control the amount of resistive force applied by the lower body apparatus to the left and right foot interfaces to provide lateral compensation for an imbalance in lateral leg strength.

12. The neurological rehabilitation system of claim 11, wherein the sub-programs that control the amount of resistive force applied by the lower body apparatus to the left and right foot interfaces to provide lateral compensation for an imbalance in lateral leg strength, comprise:
continuously measuring the amount of force being applied by the patient to the left foot interface and to the right foot interface, identify which of the patient's legs exerts more force, and designate that leg the un-impaired leg and the other leg as the impaired leg;
periodically computing the difference between a maximum force exerted by the un-impaired leg over a period of time and a maximum force exerted by the impaired leg over the period of time; and
reducing the resistance applied by the lower body apparatus to the foot interface associated with the impaired leg based on the computed force difference whenever the impaired leg is pushing on that foot interface.

13. The neurological rehabilitation system of claim 11, wherein the sub-programs that control the amount of resistive force applied by the lower body apparatus to the left and right foot interfaces to provide lateral compensation for an imbalance in lateral leg strength, comprise:
accessing a predetermined reduced resistance value associated with a patient's impaired leg; and
reducing the resistance applied by the lower body apparatus to the foot interface associated with the impaired leg by the predetermined reduced resistance value whenever the impaired leg is pushing on that foot interface.

14. The neurological rehabilitation system of claim 3, wherein the neurological rehabilitation computer program sub-program for generating the real-time visual simulation of a task that is displayed to the patient via the visual display component over time further comprises introducing a perceived risk in the task by presenting the patient with a constant flow of new challenges that require the patient to physically react with a mental urgency and importance because each new challenge is perceived as an imminent risk.

15. The neurological rehabilitation system of claim 3, further comprising a system monitor which is in two-way communication with the neurological rehabilitation controller via a wired or wireless connection, said system monitor comprising at least one display and one or more user input devices, and wherein the system monitor receives information about the neurological rehabilitation system from the neurological rehabilitation controller and is employed to input data and commands, to set parameters, make changes and add new features to the neurological rehabilitation computer program.

16. The neurological rehabilitation system of claim 3, wherein the robotic platform takes the form of a stationary human-powered recumbent quadricycle, the left foot interface takes the form of a left-side overboot, and the right foot interface takes the form of a right-side overboot, and wherein each overboot comprises:
an adjustment apparatus that secures a patient's shoe in the overboot;
a crank connection assembly that releasably attaches the overboot to a distal end of one of a pair of pedal crank arms of the lower body apparatus, and which is longitudinally adjustable in relation to an upper portion of the overboot so that the ball of the patient's foot is aligned with the distal end of the pedal crank arm during a neurological rehabilitation session; and
a pair of pivotable ankle connectors located on each side of the overboot which are in alignment with the patient's ankle, and which allow the patient to pivot their foot up and down during a neurological rehabilitation session.

17. The neurological rehabilitation system of claim 16, wherein at least one of the left-side and right-side overboots further comprises a lower leg support which provides support for the patient's lower leg to keep it from bowing in or out during a neurological rehabilitation session.

18. The neurological rehabilitation system of claim 16, further comprising at least one of a left-side and right-side synchronized support rail which provides support for the patient's leg to keep it from bowing in or out during a neurological rehabilitation session, wherein each synchronized support rail comprises:
a lower rail section and an upper rail section which are connected together at proximal ends thereof with a hinge structure, said hinge structure allowing the lower and upper rail sections to pivot in relation to each other in a single plane;
said lower rail section further comprising an ankle attachment at its distal end which attaches to the overboot adjacent the outer facing side of the patient's ankle;
said upper rail section further comprising a hip attachment at its distal end which attaches to the robotic platform adjacent the patient's hip;
said lower rail section being adjustable in length to match the length of the patient's lower leg below the knee joint, and said upper rail section being adjustable in length to match the length of the patient's upper leg above the knee joint, such that the hinge structure is aligned with the outward facing side of the patient's knee joint;
said lower rail section further comprising a lower rail section strap which is wrapped around the patient's lower leg to secure the lower leg section to the patient's lower leg; and
said upper rail section further comprising an upper rail section strap which is wrapped around the patient's upper leg to secure the upper leg section to the patient's upper leg; and wherein
whenever the synchronized support rail is attached to the overboot and robotic platform, and secured to the patient's leg, the synchronized support rail allows the patient to bend and straighten their leg in a plane substantially parallel to the plane in which the lower and upper rail sections pivot.

19. The neurological rehabilitation system of claim 3, wherein the robotic platform takes the form of a stationary human-powered recumbent quadricycle, and wherein the neurological rehabilitation system further comprises a transfer seatbelt comprising:
a seatbelt which wraps around the torso of the patient at approximately diaphragm height, and which is adjustable in circumference to fit the patient;
a plurality of transfer handles used to lift and transfer the patient as necessary onto and off of the robotic platform;
a pair of quick-disconnect latches that are attached to each side of the seatbelt and which are used to releasably secure the patient wearing the seatbelt into a seat of the robotic platform; and
at least one diaphragm sensor that senses the expansion and contraction of the patient's diaphragm as they breath and a data transfer connection that is used to transfer diaphragm sensor signals to the neurological rehabilitation controller.

20. A neurological rehabilitation system for treatment of nervous system injuries and neurological diseases, comprising:
a visual display component;
a robotic platform comprising an upper body apparatus that is contacted by a patient's left and right hands and that presents a resistive force which requires the patient to use upper body muscles to move the upper body apparatus with the patient's left and right hands;
a sensor array comprising at least one left arm sensor that measures the amount of force being applied by the patient's left arm to the upper body apparatus and at least one right arm sensor that measures the amount of force being applied by the patient's right arm to the lower body apparatus over time; and
a neurological rehabilitation controller comprising one or more computing devices, and a neurological rehabilitation computer program having a plurality of sub-programs executable by said computing device or devices, wherein the sub-programs configure said computing device or devices to,
control the amount of resistive force applied by the upper body apparatus to a left hand interface with the patient's left hand over time during a neurological rehabilitation session of the patient and a right hand interface with the patient's right hand over time during the neurological rehabilitation session of the patient, and
generate a real-time visual simulation of a task that is displayed to the patient via the visual display component over time during the neurological rehabilitation session of the patient based in part on the amount of force being applied by the patient's left and right hands to the left and right hand interfaces.

21. The neurological rehabilitation system of claim 20, the left hand interface takes the form of left-side steering blade, and the right hand interface takes the form of right-side steering blade, and wherein the resistive force applied to the left-side and right-side steering blades is applied by a steering blade servo motor of the upper body apparatus.

22. The neurological rehabilitation system of claim 21, wherein the left-side and right-side steering blades each comprise an extendable and retractable cantilevered beam that is attached at a distal end to a steering blade axle mechanism that is attached to the steering blade servo motor, and which comprises a hand grip at a distal end, each of said steering blades being operated by moving the steering blade in an up-down motion, and wherein the left-side and right-side steering blades are interlocked laterally such that when one steering blade is moved downward, the other moves upward, and wherein moving a first one of the steering blades upward and simultaneously moving the other steering blade downward simulates a turn of the robotic platform in a first direction, and wherein moving the first one of the steering blades downward and simultaneously moving the other steering blade upward simulates a turn of the robotic platform in a second direction.

23. The neurological rehabilitation system of claim 20, wherein the neurological rehabilitation computer program further comprises sub-programs that configure said computing device or devices to further control the amount of resistive force applied by the upper body apparatus to the left and right hand interfaces to provide lateral compensation for an imbalance in lateral arm strength.

24. The neurological rehabilitation system of claim 20, wherein the neurological rehabilitation computer program sub-program for generating the real-time visual simulation of a task that is displayed to the patient via the visual display component over time further comprises introducing a perceived risk in the task by presenting the patient with a constant flow of new challenges that require the patient to physically react with a mental urgency and importance because each new challenge is perceived as an imminent risk.
